(19) United States
(12) Patent Application Publication
(10) Pub. No.: US 2013/0143872 A1
(43) Pub. Date: Jun. 6, 2013

(54) INJECTABLE FORMULATION OF A REVERSE TRANSCRIPTASE INHIBITOR FOR THE TREATMENT CANCER

(71) Applicants: Nicole Stieger, Potchefstroom (ZA); Wilna Liebenberg, Potchefstroom (ZA); Melgardt Melvin De Villiers, Madison, WI (US); Mino Rodolfo Caira, Cape Town (ZA)

(72) Inventors: Nicole Stieger, Potchefstroom (ZA); Wilna Liebenberg, Potchefstroom (ZA); Melgardt Melvin De Villiers, Madison, WI (US); Mino Rodolfo Caira, Cape Town (ZA)

(21) Appl. No.: 13/696,472

(22) PCT Filed: May 31, 2011
(86) PCT No.: PCT/IB2011/052386

§ 371 Date: Feb. 19, 2013

(30) Foreign Application Priority Data
Jun. 4, 2010 (ZA) 2010/03966

(51) Int. Cl. A61K 9/00

(52) U.S. Cl. CPC A61K 9/0019
USPC 514/220

(57) ABSTRACT

This invention relates to a composition formulated into a parenteral or injectable dosage form for the treatment of cancer, more particularly, this invention relates to a composition formulated into a parenteral dosage form suitable for administration in and around cancerous tissue, the composition including: a pharmaceutically acceptable solvent including from 20% to 100% ethanol (V/V); and a reverse transcriptase inhibitor exhibiting an anti-cancer effect, dissolved in the solvent.

### INTRODUCTION AND BACKGROUND TO THE INVENTION

This invention relates to a composition formulated into a parenteral dosage form for the treatment of cancer. More particularly, this invention relates to a composition formulated into a parenteral dosage form suitable for administration in and around cancerous tissue.

Cancer is a heterogeneous disease. It manifests in a wide variety of tissue sites, with different degrees of dedifferentiation, invasiveness, and aggressiveness. Cancer cells represent dedifferentiated cells which have regained features strongly resembling stem cells, such as limitless proliferation potential and non-specific cell lineage morphology. Some of the characteristics that cancer cells adopt are the reactivation of endogenous reverse transcriptase (RT), which is originally highly expressed in embryonic and poorly differentiated cells.

Intra-tumoral injection of absolute ethanol is an established technique for the treatment of many types of cancer, including: hepatocellular carcinoma; oesophageal cancer; renal tumors, and trachea-bronchial lesions. Ethanol causes necrosis of cancerous cells, but leaves the surrounding healthy tissue and vasculature intact.

Human immunodeficiency virus type 1 (HIV-1) reverse transcriptase inhibitors (RTIs) are widely used in the clinical treatment of HIV/AIDS. Apart from their use in the treatment of HIV/AIDS, certain RTIs also exhibit anti-cancer activity.

The application of systemic RTIs via the alimentary tract is an emerging novel method of treatment in cancer therapy. The RTIs exhibit a differentiating activity in human tumor cells which is mediated by their ability to specifically reprogram gene expression. RTIs thereby restore cell function lost during tumor progression by reducing malignant cell proliferation and by promoting differentiation.

A disadvantage associated with the application of RTIs via the alimentary tract in the treatment of cancer is that it more often than not causes severe immune-mediated toxicities in individuals that are not immune-compromised. Such toxicities also occur from time to time in immune-compromised individuals.

A further disadvantage associated with some RTIs is that they are hydrophobic and range from sparingly soluble to practically insoluble, making RTIs unsuitable for parenteral dosage forms. Nevirapine is, for example, classified as practically insoluble in water with a solubility of less than 0.1 mg/ml, and efavirenz is very poorly water soluble with a solubility of 9.0 μg/ml. Adefonir is poorly water soluble at 0.4 mg/ml and telbivudine is sparingly soluble in water with a solubility of between 20 to 25 mg/ml.

### OBJECTS OF THE INVENTION

It is accordingly an object of the present invention to provide a composition formulated into a parenteral dosage form for the treatment of cancer.

It is another object of the invention to provide use of a composition formulated into a parenteral dosage form suitable for administration in and around cancerous tissue, in the treatment of cancer.

It is a further object of the invention to provide a method of formulating a composition for the treatment of cancer, into a parenteral dosage form suitable for administration in and around cancerous tissue.

It is yet a further object of the invention to provide a method of treating a patient diagnosed with cancer including the step of parenterally administering a pharmaceutically effective amount of the composition in and around cancerous tissue with which the aforesaid disadvantages may be overcome or at least minimised.

### SUMMARY OF THE INVENTION

According to a first aspect of the invention there is provided a composition for the treatment of cancer, the composition being formulated into a parenteral dosage form suitable for administration in and around cancerous tissue, the composition comprising:
a pharmaceutically acceptable solvent including from 20% to 100% ethanol (V/V); and
a reverse transcriptase inhibitor exhibiting an anti-cancer effect, dissolved in the solvent.

According to a second aspect of the invention there is provided use of a composition formulated into a parenteral dosage form suitable for administration in and around cancerous tissue, in the treatment of cancer, the composition comprising:
a pharmaceutically acceptable solvent including from 20% to 100% ethanol (V/V); and
a reverse transcriptase inhibitor exhibiting an anti-cancer effect, dissolved in the solvent.

According to a third aspect of the invention there is provided a method of formulating a composition for the treatment of cancer into a parenteral dosage form suitable for administration in and around cancerous tissue, the method including the step of dissolving a reverse transcriptase inhibitor exhibiting an anti-cancer effect in a solvent including from 20% to 100% ethanol (V/V).

The pharmaceutically acceptable solvent may be selected from a combination of ethanol with any one or more of water, propylene glycol, water-miscible adjuvants, and oil-miscible adjuvants.

The reverse transcriptase inhibitor may be selected from the groups that are sparingly soluble to practically insoluble in water. Preferably the reverse transcriptase inhibitor is selected form the group consisting of adefovir; clevudine; efavirenz; elvucitabine; nevirapine; telbivudine; and zidovudine and combinations thereof.

Preferably, but not exclusively, the reverse transcriptase inhibitor is nevirapine and the composition consists of a mixture of a pharmaceutically effective amount of nevirapine dissolved in between 20% to 100% absolute ethanol (V/V). Preferably the solvent comprises between 70% and 90% absolute ethanol and between 30% and 10% water (V/V). Further preferably, the solvent comprises 80% absolute ethanol and 20% water (V/V).

The adjuvants may be selected from the group consisting of glycerol; liquid macrogols; benzyl alcohol; polyethylene glycol; tetrahydrofurfuryl alcohol; polyethyleneglycol ether; dimethylacetamide; N-methyl-2-pyrrolidone; dimethyl sulfoxide; arachis oil; sesame oil; cotton seed oil; maize oil; almond oil; poppy seed oil; castor oil; isopropyl myristate; polyoxyethylene oleic triglycerides; ethyl oleate; benzyl benzoate and combinations thereof.

According to a fourth aspect of the invention there is provided use of a pharmaceutically effective amount of the composition formulated in accordance with the method of the third aspect of the invention, in a method of treating a patient diagnosed with cancer.

According to a fifth aspect of the invention there is provided a method of treating a patient diagnosed with cancer including the step of parenterally administering a pharmaceutically effective amount of the composition prepared in accordance with the method of the third aspect of the invention in and/or around the cancerous tissue.

### BRIEF DESCRIPTION OF THE FIGURES

The invention will now be described further, by way of non-limiting examples only, with reference to the accompanying figures wherein:

FIG. 1: is a graph depicting the change in nevirapine solubility (at 25 degrees Celsius) versus ethanol fraction in a water-ethanol solvent system (horizontal axis: weight fraction ethanol; vertical axis: solubility (mg/ml));

FIG. 2: is a graph depicting the correlation between the reduction in water activity and the solubility of nevirapine in these ethanol-water mixtures (horizontal axis: moles water added; left vertical axis: reduction in water activity (%); right vertical axis: solubility (mg/ml));

FIG. 3: is a solubility curve of nevirapine as a function of water activity at 25 degrees Celsius (horizontal axis: water activity; vertical axis: solubility (mg/ml)); and

FIGS. 4 and 5: are solubility diagrams of nevirapine in mixtures of ethanol and water. The crystal forms and crystal habits of recrystallisation products obtained from saturated solvent mixtures are shown.

### DESCRIPTION OF PREFERRED EMBODIMENTS OF THE INVENTION

According to a preferred embodiment of the invention there is provided a composition formulated into a parenteral dosage form suitable for administration in and around cancerous tissue in the treatment of cancer.

The parenteral composition comprises a pharmaceutically acceptable solvent including from 20% to 100% ethanol (V/V); and a reverse transcriptase inhibitor exhibiting an anti-cancer effect, dissolved in the solvent. Preferably, the solvent includes ethanol in combination with any one or more of water, propylene glycol, water-miscible adjuvants, and oil-miscible adjuvants.

The reverse transcriptase inhibitor is selected from the groups that are sparingly soluble to practically insoluble in water and include adefovir; clevudine; efavirenz; elvucitabine; nevirapine; telbivudine; and zidovudine and combinations thereof.

The adjuvants are selected from the group consisting of glycerol; liquid macrogols; benzyl alcohol; polyethylene glycol; tetrahydrofurfuryl alcohol; polyethyleneglycol ether; dimethylacetamide; N-methyl-2-pyrrolidone; dimethyl sulfoxide; arachis oil; sesame oil; cotton seed oil; maize oil; almond oil; poppy seed oil; castor oil; isopropyl myristate; polyoxyethylene oleic triglycerides; ethyl oleate; benzyl benzoate; and combinations thereof.

### Preparation of the Parenteral Composition

The first step in the preparation of the parenteral composition, according to the first embodiment of the invention, is to dissolve nevirapine in polymorphic, amorphous, solvated or desolvated form in ethanol by agitation and optional heating. The nevirapine used is preferably the conventional anhydrous form (Form I) or alternatively nevirapine nano- /microspheres.

It was found that the approximate maximum solubility of nevirapine, in ethanol at 75 degrees Celsius, is 1 g per 45 ml.

The second step in the preparation of the parenteral composition is, once the nevirapine is fully dissolved in ethanol, to gradually add a volume of water ranging from 0% to 80% (V/V). Nevirapine is dissolved in between 20% to 100% absolute ethanol (V/V). Preferably the solvent comprises between 70% and 90% absolute ethanol and between 30% and 10% water (V/V). Further preferably, the solvent comprises 80% absolute ethanol and 20% water (V/V).

The third, but optional, step in the preparation of the parenteral composition is the addition of one or more adjuvants selected from the above list to further stabilise the solution and to prevent crystallisation of the nevirapine.

### Analysis of the Results

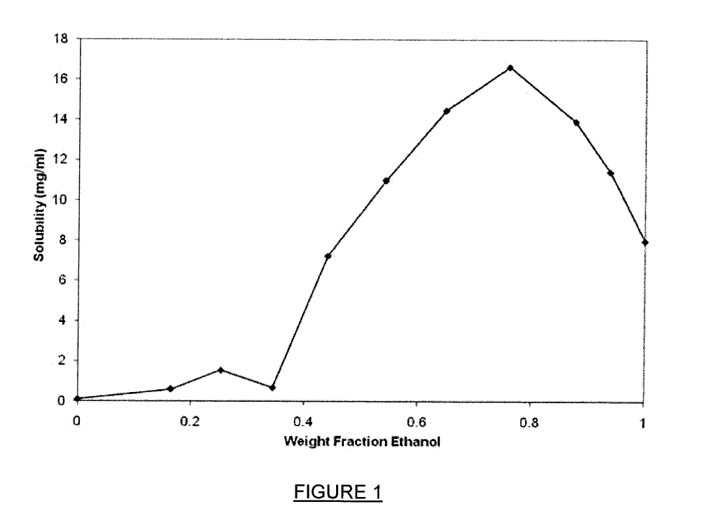

The solubility of nevirapine in water, ethanol and in a water-ethanol co-solvent system was determined. It was found that the solubility of commercially available anhydrous nevirapine (Form I) in water (at 25 degrees Celsius) is less than 0.1 mg/ml and its solubility in absolute ethanol is 8.0 mg/ml. An unexpected dramatic increase in solubility was observed with the addition of ethanol to a water system at an ethanol weight fraction of above 0.4 (40% ethanol, 60% water) as illustrated in FIG. 1.

At an ethanol weight fraction of 0.80 (FIG. 1) (80% absolute ethanol and 20% water (V/V)), the maximum solubility of nevirapine is 16.6 mg/ml, followed by a decrease with the addition of more ethanol.

It was surprisingly and unexpectedly found that nevirapine with an ethanol weight fraction of 0.80 displays a 166 fold increase in solubility over the solubility of nevirapine in pure water.

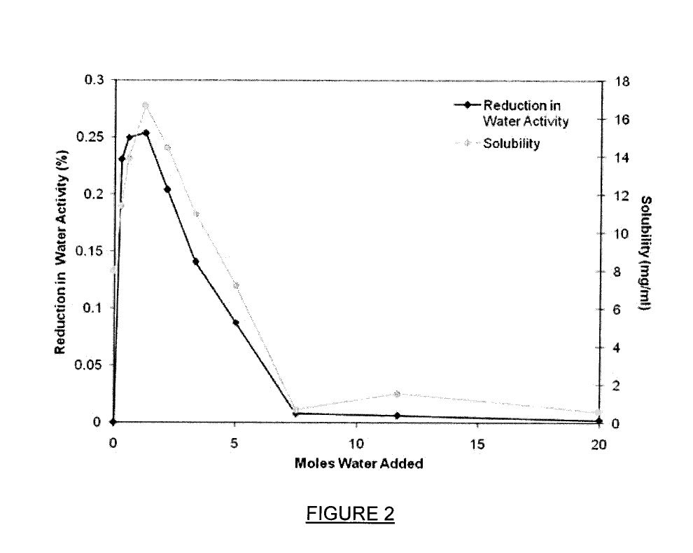

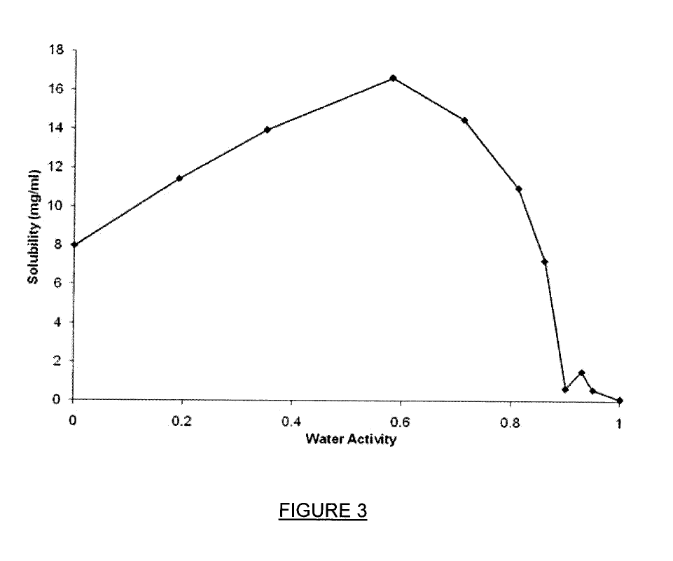

Referring to FIGS. 2 and 3 it was further found that the change in solubility of nevirapine is linked to the change in water activity in the solvent systems. Water activity is reduced by adding an increased amount of water to a constant volume of ethanol. The ethanol reduces the water activity of the aqueous phases and in turn changes the solubility of nevirapine, followed by crystal formation that precipitates at different water-ethanol concentrations. This reduction in water activity and the solubility of the nevirapine in the ethanol-water co-solvent system are illustrated in FIG. 2.

It is therefore asserted that there is direct correlation between the reduction in water activity, and the solubility of nevirapine in ethanol-water mixtures. Once the phase boundaries in one co-solvent-water system have been established, the activity boundaries of other co-solvent-water systems are simply converted to water concentrations, resulting in considerable resources being saved by expressing phase equilibria in anhydrous-hydrate systems in terms of water activity.

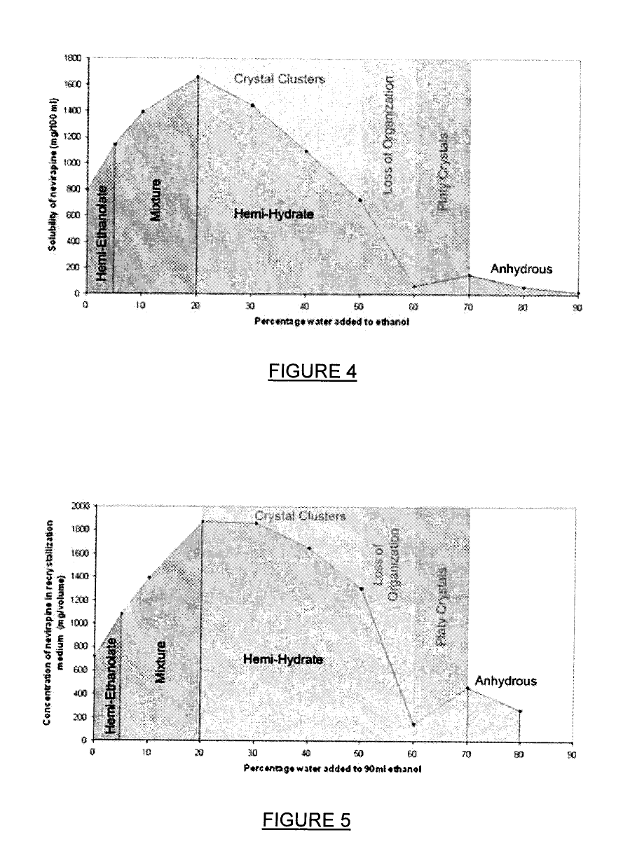

The solubility and crystal formation that precipitates at different water-ethanol concentrations of nevirapine are further illustrated in FIGS. 4 and 5 and table 1.

Table 1 is a summary of the influence of water-ethanol co-solvent systems on the crystal forms and crystal habits of nevirapine. The amount of anhydrous nevirapine dissolved in each solvent system was 2 g±0.01 g throughout.

TABLE 1

| Recrystallization Medium | | | |
|---|---|---|---|
| | Approximate | | |
| EtOH:H₂O | Water Content | Nevirapine Form | Crystal |
| (ml) | (% v/v) | Obtained | Habit |
| 90:0 | 0 | Hemi-Ethanolate | Tabular crystals |
| 90:4.7 | 5 | Hemi-Ethanolate | Smaller tabular |
| | | | crystals |
| 90:0 | 10 | Mixture of | Very small tabular |
| | | Anhydrous, Hemi- | and columnar |
| | | Ethanolate and | crystals |
| | | Hemi-Hydrate | |
| 90:22.5 | 20 | Hemi-Hydrate | Disorganized |
| | | | columnar clusters |
| 90:38.6 | 30 | Hemi-Hydrate | Disorganized |
| | | | columnar clusters |
| 90:60 | 40 | Hemi-Hydrate | Columnar clusters |
| 90:90 | 50 | Hemi-Hydrate | Platy clusters |
| 90:135 | 60 | Hemi-Hydrate | Platy crystals |
| | | | with rudimentary |
| | | | clustering |
| 90:210 | 70 | Hemi-Hydrate | Platy crystals |
| 90:360 | 80 | Anhydrous | Very small bladed |
| | | | crystals |

FIG. 4 illustrates the solubility of nevirapine (mg/100 ml at 25 degrees Celsius) and the percentage (V/V) of water added to ethanol. FIG. 5 illustrates the concentration of nevirapine in a recrystallisation medium and the percentage of water added to 90 ml ethanol. Both figures are therefore indicative of the relationship between the solubility and concentration of nevirapine, the solvent composition and the effect that the relationship has on the crystal forms obtained.

It is evident from FIGS. 4 and 5 that the solubility of nevirapine did not decrease smoothly as increased amounts of water were added.

The composition is formulated in any one of the following injectable dosage forms selected from a group consisting of intra-tumoral-, subcutaneous-, intramuscular-, intradermal-, intracavernous- and percutaneous injections; intravesical infusion and limb-, tissue- and organ perfusions.

The prepared parenteral composition according to the invention is injected through the skin (percutaneously) into the cancerous tumor of the patient with the assistance of ultrasound or CT visual guidance. The composition induces tumor destruction by drawing water out of cancerous tumor cells and thereby denaturing the structure of cellular proteins. The composition further reduces malignant cell proliferation and promotes differentiation. Should cancerous tissue be encountered during an operation, the parenteral composition is injected directly into the cancerous tissue.

The administration of the parenteral composition according to the invention into and around cancerous tissue results in the delivery of relatively high concentrations of the active ingredient (RTI) directly to the cancerous tissue whilst reducing the side-effects and systemic immune-mediated toxicity of RTIs, in turn alleviating the disadvantage of severe immune-mediated toxicities experienced in individuals.

It will be appreciated that in terms of the invention, variations in details in the composition formulated into a parenteral dosage form for the treatment of cancer and more particularly relating to a composition formulated into a parenteral dosage form suitable for administration in and around cancerous tissue, are possible without departing from the scope of the appended claims.

1. A composition for the treatment of cancer, the composition being formulated into a parenteral dosage form suitable for administration in and around cancerous tissue, the composition comprising a pharmaceutically acceptable solvent including from 20% to 100% ethanol (V V); and a reverse transcriptase inhibitor exhibiting an anti-cancer effect, dissolved in the solvent.

2. A composition according to claim 1 wherein the pharmaceutically acceptable solvent includes a combination of ethanol with any one or more of water, propylene glycol, water-miscible adjuvants, and oil-miscible adjuvants.

3. A composition according to claim 1 wherein the reverse transcriptase inhibitor is selected from the groups that are sparingly soluble to practically insoluble in water.

4. A composition according to claim 3 wherein the reverse transcriptase inhibitor is selected from the group consisting of adefovir; clevudine; efavirenz; elvucitabine; nevirapine; telbivudine; and zidovudine and combinations thereof.

5. A composition according to claim 4 wherein the reverse transcriptase inhibitor is nevirapine.

6. A composition according to claim 5 consisting of a mixture of a pharmaceutically effective amount of nevirapine dissolved in between 20% to 00% absolute ethanol (V/V).

7. A composition according to claim 6 wherein the solvent comprises between 70% and 90% absolute ethanol and between 30% and 10% water (V/V).

8. A composition according to claim 7 wherein the solvent comprises 80% absolute ethanol and 20% water (V/V).

9. A composition according to claim 2 wherein the adjuvants are selected from the group consisting of glycerol; liquid macrogols; benzyl alcohol; polyethylene glycol; tetrahydrofurfuryl alcohol; polyethyleneglycol ether; dimethylacetamide; N-methyl-2- pyrrolidone; dimethyl sulfoxide; arachis oil; sesame oil; cotton seed oil; maize oil; almond oil; poppy seed oil; castor oil; isopropyl myristate; polyoxyethylene oleic triglycerides; ethyl oleate; benzyl benzoate and combinations thereof.

10. Use of a composition formulated into a parenteral dosage form suitable for administration in and around cancerous tissue, in the treatment of cancer, the composition comprising a pharmaceutically acceptable solvent including from 20% to 100% ethanol (V/V); and a reverse transcriptase inhibitor exhibiting an anti-cancer effect, dissolved in the solvent.

11. A method of formulating a composition for the treatment of cancer into a parenteral dosage form suitable for administration in and around cancerous tissue, the method including the step of dissolving a reverse transcriptase inhibitor exhibiting an anti-cancer effect in a pharmaceutically acceptable solvent including from 20% to 100% ethanol (V/V).

12. Use of a pharmaceutically effective amount of the parenteral composition according to claim 1 in a method of treating a patient diagnosed with cancer.

13. A method of treating a patient diagnosed with cancer including the step of parenterally administering a pharmaceutically effective amount of the composition according to claim 1 in and/or around the cancerous tissue.

14. A composition for the treatment of cancer substantially as herein described and exemplified with reference to the accompanying figures.
